(19) United States
(12) Patent Application Publication
(10) Pub. No.: US 2024/0023731 A1
(43) Pub. Date: Jan. 25, 2024

(54) PILLOW, BLANKET, TOPPER, MATTRESS AND/OR OVERLAY FOR CALMING A USER'S AUTONOMIC NERVOUS SYSTEM

(71) Applicants: NATURECOM TEC UG (haftungsbeschränkt), Altenstadt (DE); ZWEIZEHN GMBH & CO. KG, Püchersreuth (DE)

(72) Inventors: Gerhard ADAM, Püchersreuth (DE); Lars ENGEL, Schirmitz (DE); Dave Schimurda, Altenstadt (DE); Martin SCHWAB, Schirmitz (DE)

(73) Assignees: NATURECOM TEC UG (haftungsbeschränkt), Altenstadt (DE); ZWEIZEHN GMBH & CO. KG, Püchersreuth (DE)

(21) Appl. No.: 18/224,955

(22) Filed: Jul. 21, 2023

(30) Foreign Application Priority Data
Jul. 22, 2022 (DE) 102022118391.2

(51) Int. Cl. A47G 9/00; A61M 21/02; A61K 31/00; A61K 36/185

(52) U.S. Cl. CPC A47G 9/007; A61M 21/02; A61K 31/658; A61K 36/185; A61M 2021/0077

(57) ABSTRACT

A pillow, blanket, topper, mattress and/or overlay for calming the autonomic nervous system of a user includes at least one filling chamber within which at least one filling material is placed and at least one filling region within the filling chamber. The filling region is formed by at least one interstitial space which is at least partially bounded by the filling material. The interstitial space is at least partially formed by and/or at least partially formed with a cannabinoid-containing material and/or a THC-containing material, wherein the filling material of the filling chamber is substantially free of the cannabinoid-containing material and/or a THC-containing material.

The present invention relates to a pillow, blanket, topper, mattress and/or overlay for calming a vegetative nervous system of a user, and to a method of a pillow, blanket, topper, mattress and/or overlay for calming a vegetative nervous system of a user according to the generic terms of the respective claims 1 and 10.

According to the invention, the pillow, blanket, topper, mattress and/or overlay for calming a vegetative nervous system of a user comprises at least one filling chamber within which at least one filling material is introduced and at least one filling region within the filling chamber, wherein the filling region is formed by at least one intermediate space which is at least partially delimited by the filling material. The interstitial space is formed at least partially by a cannabinoid-containing material and/or by a THC-containing material and/or is formed of dried useful hemp with its natural, original active ingredients and/or at least partially with said material, wherein the filling material of the filling chamber is substantially free of the cannabinoid-containing material and/or by a THC-containing material.

The THC active substance content described here can be between at least 0.001% and at most 5%, preferably between at least 0.15% and 0.5%, for example below 0.2% or 0.3%.

According to at least one embodiment, the filling region is completely surrounded by the filling chamber.

According to at least one embodiment, the material is formed with cannabis plant flowers.

According to at least one embodiment, the material is formed with a hemp containing THC or a cannabinoid.

According to at least one embodiment, the hemp comprises whole hemp plants, wherein the hemp plant comprises at least the shoot axis comprising a flower stalk, petals and a flower itself.

According to at least one embodiment, the filling material is at least partially a filling layer material comprising a plurality of adjacent layers, in particular a quilted fabric, wherein between at least two adjacent layers of the filling material the material is at least partially introduced and thus between two and/or through the two adjacent layers the filling area is at least partially formed.

The following are possible pillow fillings:

Loose pillow fillings dominate the pillow market and are especially popular with people who like to scrunch their pillows and have a high need for comfort. The advantage of these pillows is that they are very easy to push into the desired shape.

Pillows with a filling of down and feathers are the classic and many people do not know or want any other filling. Understandably, after all, the combination of supportive feathers and soft down is ideal for high-quality pillows.

A somewhat more sophisticated variant of the feather/down pillow is the three-chamber pillow. With these pillows, the two filling materials are processed separately in the pillow. The supportive feathers are housed in the middle layer, while the soft down in the outer layers provides soft comfort.

Hollow fibre is a synthetic filling material that is very common in pillows. The modern and airy material is usually made of polyester and is a good alternative for people who are looking for an animal-free alternative to feather pillows.

Pillows filled with hollow fibre often have a very high volume and impress with their elasticity, high life expectancy and dimensional stability even after frequent washing. Another advantage is the strong breathability that results from the hollow spaces in the beads. Heat and moisture can thus be quickly transported away, but the pillows still keep you warm.

Padded fillings, on the other hand, are ultra-light and hardly noticeable, so that this effect is maintained for as long as possible, the wadding is usually spun into a kind of fleece. The padding in pillows is usually made of polyester or polyurethane, but natural padding made of lyocell or bamboo viscose is also increasingly found. Pillows filled with cotton wool or microfibre are very soft. You can therefore sink deeply into the pillows, but they have very little support.

Flakes or also rods are a very exciting filling and a great alternative to a firm foam pillow. Pillows filled with flakes offer high support, are dimensionally stable and, depending on what kind of flakes are used, very adaptable. Flakes made of visco foam or gel foam, for example, are actually used in neck support pillows and are the go-to filling for neck problems.

The second variant would be flakes of PU foam or cold foam. Both foams offer enormous support, but are not quite as adaptable as latex foam, visco foam or gel foam.

Pillows filled with spelt or millet may not be among the most popular pillows, but they certainly have their raison d′être. However, it is often claimed that these pillows or fillings do not provide sufficient support. If there is not enough dinekl fur in the pillow, the filling slides back and forth and may be displaced from the head. However, if there is enough filling, these pillows provide excellent support. In addition, the pillows have a light but pleasantly warming effect and a slight rippling sensation when you turn around.

For most people, these fillings are out of the question, but they do exist and should be mentioned. Pillows with animal hair filling are usually filled with either horsehair, virgin wool or camel fluff. A horsehair fleece is very flat and firm. Pure new wool and also camel fluff are somewhat softer and more adaptable. Thanks to their almost legendary ability to wick away moisture particularly well, these pillows are found almost exclusively in cases of heavy perspiration.

The supportive effect and pressure relief of viscoelastic foam is unbeaten. At first glance, these pillows often appear very firm, but they change shape under the influence of body heat and adapt ideally to the head and neck.

Moulded foam is suitable for the user who is looking for a dimensionally stable pillow that relieves the strain on your neck. It should be noted, however, that the room in which the pillow is used should not have a room temperature below 18° C., as the pillow then takes a long time to assume its initial shape when the user turns during sleep.

Although gel foam is somewhat softer than memory foam, it offers very similar properties and adapts to a new sleeping position considerably faster. Another advantage is the open-pored structure of gel foam. While Visco foam tends to accumulate body heat, gel foam is much better at dissipating sweat and heat. Thus, gel foam offers good support and high comfort, reacts faster and is completely independent of the room temperature.

Pillows with a latex filling feel somewhat softer than pillows made of viscoelastic foam and the material reacts more quickly to changes in the lying position. You can only influence the degree of hardness of latex pillows if you pay attention to whether the pillow is made of synthetic or natural latex. On the one hand, natural latex is a purely natural product and also somewhat softer than synthetically produced latex.

According to at least one embodiment, the material is loosely inserted between adjacent layers.

According to at least one embodiment, the material is placed in pockets of the filling chamber, the pockets being delimited by chamber walls separating the material from the filling material.

According to the invention, in the method of a pillow, blanket, topper, mattress and/or overlay for calming a vegetative nervous system of a user, at least one filling chamber, within which at least one filling material is introduced, is created and, in a further step, at least one filling region is created within the filling chamber, the filling region being formed by at least one intermediate space which is at least partially delimited by the filling material.

In this case, the intermediate space is at least partially formed by a cannabinoid-containing material and/or by a THC-containing material and/or is at least partially formed with this material, the filling material of the filling chamber being substantially free of the cannabinoid-containing material and/or by a THC-containing material.

In the following, the invention described herein will be explained in more detail with reference to a FIGURE.

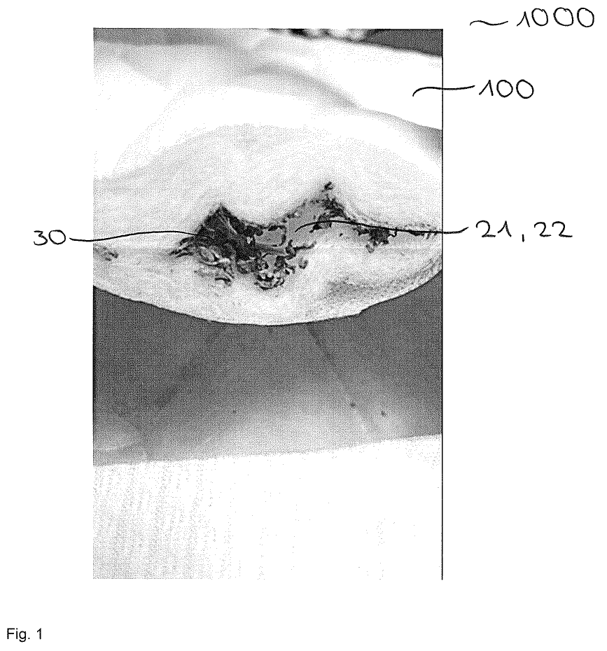

FIG. 1 shows a pillow, blanket, topper, mattress and/or overlay 100 for calming a vegetative nervous system of a user, comprising at least one filling chamber 21 within which at least one filling material 30 is placed, and at least one filling area 22 within the filling chamber 21, wherein the filling area 22 is formed by at least one intermediate space which is at least partially delimited by the filling material 30.

The interstitial space is thereby at least partially formed by and/or at least partially formed with a cannabinoid-containing material and/or a THC-containing material, wherein the filling material 30 of the filling chamber 21 is substantially free of the cannabinoid-containing material and/or by a THC-containing material.

The material is formed with cannabis plant flowers and/or with a hemp containing THC or a cannabinoid. However, the material may also omit cannabis flowers altogether, particularly within manufacturing tolerance. The hemp may consist of whole hemp plants, the hemp plant comprising at least the shoot axis comprising a flower stalk and/or petals and/or a flower itself.

Furthermore, it can be seen that the filling material 30 is at least partially a filling layer material comprising a plurality of layers adjacent to each other, in particular a quilted fabric, wherein between at least two adjacent layers of the filling material 30 the material is at least partially introduced and thus between two and/or through the two adjacent layers the filling area 22 is at least partially formed.

In this case, the material is loosely inserted between adjacent layers and placed in pockets of the filling chamber 21, the pockets being delimited by chamber walls which separate the material from the filling material 30.

FIG. 1 thus also shows a method 1000 of a cushion, blanket, topper, mattress and/or overlay 100 for calming a vegetative nervous system of a user, wherein at least one filling chamber 21, within which at least one filling material 30 is introduced, is created and, in a further step, at least one filling region 22 is created within the filling chamber 21, the filling region 22 being formed by at least one intermediate space which is at least partially delimited by the filling material 30.

The interstitial space is thereby at least partially formed by and/or at least partially formed with a cannabinoid-containing material and/or a THC-containing material, wherein the filling material 30 of the filling chamber 21 is substantially free of the cannabinoid-containing material and/or by a THC-containing material.

### LIST OF REFERENCE SIGNS

21 Filling chamber
22 Filling area
30 Filling material
100 Pillow, blanket, topper, mattress and/or overlay
1000 Process

1. A pillow, blanket, topper, mattress and/or overlay (100) for calming a vegetative nervous system of a user, comprising
at least one filling chamber (21) within which at least one filling material (30) is placed
at least one filling region (22) within the filling chamber (21), the filling region (22) being formed by at least one intermediate space which is at least partially delimited by the filling material (30),
characterised in that
said interspace is at least partially formed by a cannabinoid-containing material and/or by a THC-containing material and/or is formed of dried useful hemp with its natural, original active ingredients and/or at least partially with said material, wherein the filling material (30) of the filling chamber (21) is substantially free of said cannabinoid-containing material and/or by a THC-containing material.

2. The pillow, blanket, topper, mattress and/or overlay (100) according to claim 1, characterized in that
the filling area (22) is completely surrounded by the filling chamber (21).

3. The pillow, blanket, topper, mattress and/or overlay (100) according to claim 1,
characterized in that
the material is formed with cannabis plant flowers.

4. The pillow, blanket, topper, mattress and/or overlay (100) according to claim 1,
characterised in that
the material is formed with a hemp containing THC or a cannabinoid.

5. The pillow, blanket, topper, mattress and/or overlay (100) according to claim 1,
characterized in that
the hemp consists of whole hemp plants, the hemp plant comprising at least the shoot axis consisting of a flower stalk, petals and a flower itself.

6. The pillow, blanket, topper, mattress and/or overlay (100) according to claim 1,
characterised in that
the filling material (30) is at least partly a filling layer material comprising a plurality of mutually adjacent layers, in particular a quilted fabric, wherein between at least two mutually adjacent layers of the filling material (30) the material is at least partly introduced and thus between two and/or through the two adjacent layers the filling region (22) is at least partly formed.

7. The pillow, blanket, topper, mattress and/or overlay (100) according to claim 1,
characterised in that
the material is loosely inserted between adjacent layers.

8. The pillow, blanket, topper, mattress and/or overlay (100) according to claim 1,
characterized in that
the material is placed in pockets of the filling chamber (21), the pockets being delimited by chamber walls separating the material from the filling material (30).

9. A method (1000) of a pillow, blanket, topper, mattress and/or overlay (100) for calming a vegetative nervous system of a user according to claim 1,
wherein
at least one filling chamber (21), within which at least one filling material (30) is introduced, is created, and in a further step
at least one filling region (22) is created within the filling chamber (21), the filling region (22) being formed by at least one intermediate space which is at least partially delimited by the filling material (30),
characterised in that
the interstitial space is at least partially formed by and/or at least partially formed with a cannabinoid-containing material and/or a THC-containing material, the filling material (30) of the filling chamber (21) being substantially free of the cannabinoid-containing material and/or by a THC-containing material.
